(19) United States
(12) Patent Application Publication
(10) Pub. No.: US 2008/0208296 A1
(43) Pub. Date: Aug. 28, 2008

(54) APPARATUS AND METHODS FOR TREATING CELLULITE BY COMBINING EXERCISE WITH THE APPLICATION OF LIGHT ENERGY

(76) Inventors: Gary H. Smith, Salt Lake City, UT (US); Doug Smith, Sandy, UT (US); Scot N. Andersen, Draper, UT (US)

Correspondence Address: WORKMAN NYDEGGER, 60 EAST SOUTH TEMPLE, 1000 EAGLE GATE TOWER, SALT LAKE CITY, UT, 84111, US

(21) Appl. No.: 11/551,602

(22) Filed: Oct. 20, 2006

Related U.S. Application Data
(60) Provisional application No. 60/729,060, filed on Oct. 20, 2005.

(51) Int. Cl. A61N 5/06; A63B 26/00

(52) U.S. Cl. 607/89; 482/1

(57) ABSTRACT

Exercise by a person coupled with illuminating cellulite-containing tissue of a person with light produced by a semi-conductor light source reduces cellulite in such tissue. Methods and systems for reducing cellulite may employ light from a light source comprising a semi-conductor material coupled with exercise equipment. Clothing may be provided that incorporates the light source. Clothing may alternatively have increased transmissiveness in areas adjacent to cellulite-containing tissue. A light activating composition may increase the effectiveness of cellulite reduction.

### RELATED APPLICATIONS

The present application claims the benefit of U.S. Provisional Patent Application Ser. No. 60/729,060, filed Oct. 20, 2005, the disclosure of which is incorporated by reference in its entirety.

### BACKGROUND OF THE INVENTION

1. The Field of the Invention

The invention relates to apparatus and methods for reducing cellulite in a person's body. The apparatus and methods disclosed herein can include the combination of radiant energy and physical exercise, optionally using a photo-activating cream, to reduce cellulite.

2. Related Technology

Appearance, and in particular the appearance of cellulite, is of great concern for many people, and in particular, of for women, who tend to develop cellulite more than men and who are more likely to be scrutinized according to their outward beauty given societal norms. Cellulite refers to deposits of subcutaneous fat within fibrous connective tissue that give a puckered or dimpled appearance to the skin surface. These columns of fat cells can be exposed resulting in visible dimples or wrinkles referred to as “cellulite”. Cellulite is most often found in the on the thighs, buttocks, and hips of women. Various industries, such as the health and cosmetic industries, have offered a wide variety remedies in the form of both physical and chemical treatments, which have claimed various levels of effectiveness in reducing cellulite.

The term “cellulite”, as used herein, refers to the physical conditions of cellulite and/or the appearance of cellulite. Some examples of conventional treatments used for reducing cellulite include liposuction, intensive massage (e.g., often using suction), ultrasonic treatments, and certain radiant light treatments, as well as topical firming, debrading, desensitizing, sensitizing, exfoliating, and stimulating creams and moisturizers. Some treatments have also included several of these treatments in various combinations and formats.

The term “cellulite-containing tissue” means tissue of a person that is characterized as having visible pock marts of cellulite tissue.

One product, called the “Cellubike” by a company named Logicam, combines exercise with treatments of radiant light for reducing cellulite. As shown in FIG. 1, the Cellubike 100 uses infrared radiation in combination with a stationary exercise bicycle 105 to treat cellulite while a person is exercising on the stationary bicycle 105. The Cellubike 100 incorporates rows of lamps 110 in a substantially circular shaped chamber 120, much like a magnetic resonance imaging (MRI) machine, to provide infrared light and thermal heat to parts of the person's body performing the exercise. To provide this infrared radiation, the Cellubike 100 has incorporated these rows of lamps 110 (i.e., halogen lamps) into a substantially bulky, inefficient, costly to manufacture and operate, and uncomfortable design for the person performing the exercise. These lamps 110 create a large amount of heat and consume a relatively high quantity of electrical energy. Moreover, the areas of the body where cellulite is most often found (i.e., thighs, buttocks and hips) are at least partially inaccessible to the light energy because of the sitting and hunched over position of the person performing the exercise.

As shown in FIG. 1, the light providing chamber 120 is large to accommodate the lamps. The light providing chamber 120 is also formed integral to the stationary exercise bicycle 105 creating a bulky, uncomfortable, and awkward exercising apparatus. Because the light providing chamber 120 of the Cellubike is integrated with the stationary exercise bicycle 105, the Cellubike 100 requires a substantial investment in a single exercise apparatus, thus reducing flexibility in the types of exercises that a person would otherwise be allowed to perform.

As a result of these and other shortcomings, there is still a need in the cosmetic and health industries for improved remedies and systems for reducing cellulite and/or the appearance of cellulite.

### BRIEF SUMMARY OF THE PREFERRED EMBODIMENTS

According to one aspect of the invention, a method for reducing cellulite comprises performing an exercise and illuminating cellulite-containing tissue using light produced by a light source comprising a semiconductor material. The exercise may be performed using exercise equipment, for example, a treadmill, a stationary bicycle, a stair stepping device, an elliptical glider, a free weight exercise device, and/or a resistance weight exercise device. The light source may comprise at least one of a light source comprising silicon, a laser or a laser diode. The light may advantageously have multiple substantially defined spectra.

A person performing the exercise may wear specially designed clothing comprising a first portion including a first material and a second portion adjacent to the cellulite-containing tissue and including a second material, wherein the second material is more transmissive to the light than the first material. Alternatively, the clothing may comprise a removed portion adjacent to the cellulite-containing tissue. The clothing may actual comprise at least a portion of the light source used to illuminate the cellulite-containing tissue.

The method may further comprise applying to skin of the cellulite-containing tissue a composition that increases reception of the light. The composition may comprise at least one of a colorant, a pigment, a chemical receptor, methylenediaminetetraacetic acid (EDTA), or a selected pH for enhancing reception of of the light.

The light source may be fixedly or removably attached to a piece of equipment for performing the exercise or it may be separate from the piece of equipment for performing the exercise. The light source may be coupled to, or incorporated with, clothing worn adjacent to the cellulite-containing tissue.

A system for reducing cellulite may comprise means for performing the methods described herein (e.g., as recited in claim 1). The system typically includes exercise equipment, such as a treadmill, a stationary bicycle, a stair stepping device, an elliptical glider, a free weight exercise device, or a resistance weight exercise device. The exercise equipment may advantageously be configured such that a person exercising using the exercise equipment is exercising in a substantially upright position such that the person's thighs, buttocks, and hips are substantially unobstructed from illumination by the light source.

Clothing for use in reducing cellulite may comprise a fabric and a light source comprising a semiconductor material, attached to the fabric, and configured to illuminate cellulite-containing tissue of a person wearing the clothing. The light source typically comprises an array of light sources incorporated with, or attached to, the clothing. The array of light sources may be located within the clothing to illuminate the thigh, buttocks, or hips areas of a person wearing the clothing. The clothing may further comprise an activation composition incorporated into the fabric.

A kit for use in reducing cellulite in a person may comprise a light source comprising a semiconductor material and an electro-muscle stimulation device. Alternatively, a kit for use in reducing cellulite in a person may comprise a light source comprising a semiconductor material and a light activating composition for application to a person's body for enhancing activity of light emitted by the light source when it contacts the person's body.

### BRIEF DESCRIPTION OF THE DRAWINGS

FIG. 1 is an illustration of the Cellubike;

FIG. 2 illustrates a stationary exercise bicycle and a person exercising using the stationary exercise bicycle;

FIGS. 3A, 3B, and 3C illustrate three examples of exercises where the synergistic effects of exercise and light for reduction of cellulite can be more fully realized than in FIG. 2;

FIG. 4 illustrates a treadmill that is releasably attached to a light producing structure;

FIG. 5 illustrates tissue as it is illuminated by multiple light sources arranged in a vertical array;

FIG. 6 illustrates a piece of clothing where the fabric of the clothing is made from a material that is selected for its light transmissive properties;

FIG. 7 illustrates clothing made from two materials having different light transmissive properties;

FIG. 8 illustrates clothing where portions of the clothing are removed to expose that portion of the body;

FIG. 9 illustrates clothing incorporating arrays of light sources;

FIG. 10 illustrates exercise equipment incorporating light sources;

FIG. 11 is a block diagram illustrating a method for reducing cellulite;

FIG. 12 is a block diagram illustrating a method for reducing cellulite; and

FIG. 13 is a block diagram illustrating a method for reducing cellulite.

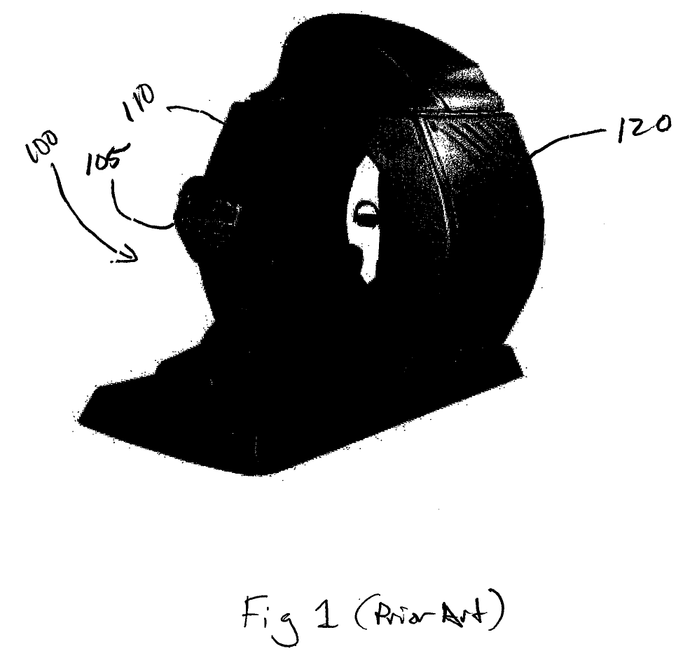

### DETAILED DESCRIPTION OF THE PREFERRED EMBODIMENTS

The embodiments discussed herein relate to apparatus and methods for reducing cellulite and/or the appearance of cellulite. The embodiments disclosed herein are described by the structure and operation of the various examples illustrated. It should be understood that the drawings are diagrammatic and schematic representations of such example embodiments and, accordingly, are not limiting of the scope of the embodiments, nor are the drawings necessarily drawn to scale. Well-known devices and processes have been excluded from this disclosure so as not to obscure the discussion in details that would otherwise be known to one of ordinary skill in the art.

Several embodiments incorporate radiant energy, such as light, along with exercise in more efficient, cost effective, treatment effective, flexible, robust, and/or ergonomic designs. Several embodiments also incorporate radiant energy with light-activation compositions for improving the delivery of the radiant energy to the areas of the patient's body requiring more intense treatment. As referred to herein, the “patient” is the person whose cellulite is to be reduced. Several other embodiments combine these and other treatments in various new combinations and configurations, which can produce many unexpected and/or synergistic results from the particular combination and/or configuration. The term “light,” as used herein, refers to electromagnetic radiation of any wavelength. Thus, the term “light” includes visible, invisible, infrared, and ultraviolet radiation unless otherwise noted.

### I. Light and Exercise Treaments

Application of light during exercise can have several benefits for reducing cellulite. For example, application of light can result in increased blood flow, stimulation of connective tissue, improved healing of muscles, other stimulation of tissue at the cellular level, introduction of Vitamin D, emotional benefits, as well as other benefits. Exercise is also beneficial for reducing cellulite. For example, exercise also increases blood flow, burns fat, and strengthens and stimulates connective tissue. Several embodiments relate to a combination of light treatments with exercise to produce a synergistic effect to reduce cellulite.

A. Selection of Exercise Equipment for Access to Target Areas

An aspect of several embodiments relates to the selection of exercises (and exercise equipment) for providing maximum light exposure to the areas of the body where cellulite is most often found. Many types of machines and systems are currently used for exercise. To name a few examples, there are exercise bicycles, treadmills, stair stepping machines, free weight machines, resistance weight machines, and elliptical gliding exercise machines. Light treatments for reducing cellulite can be applied during use of any exercise machine according to several embodiments disclosed herein.

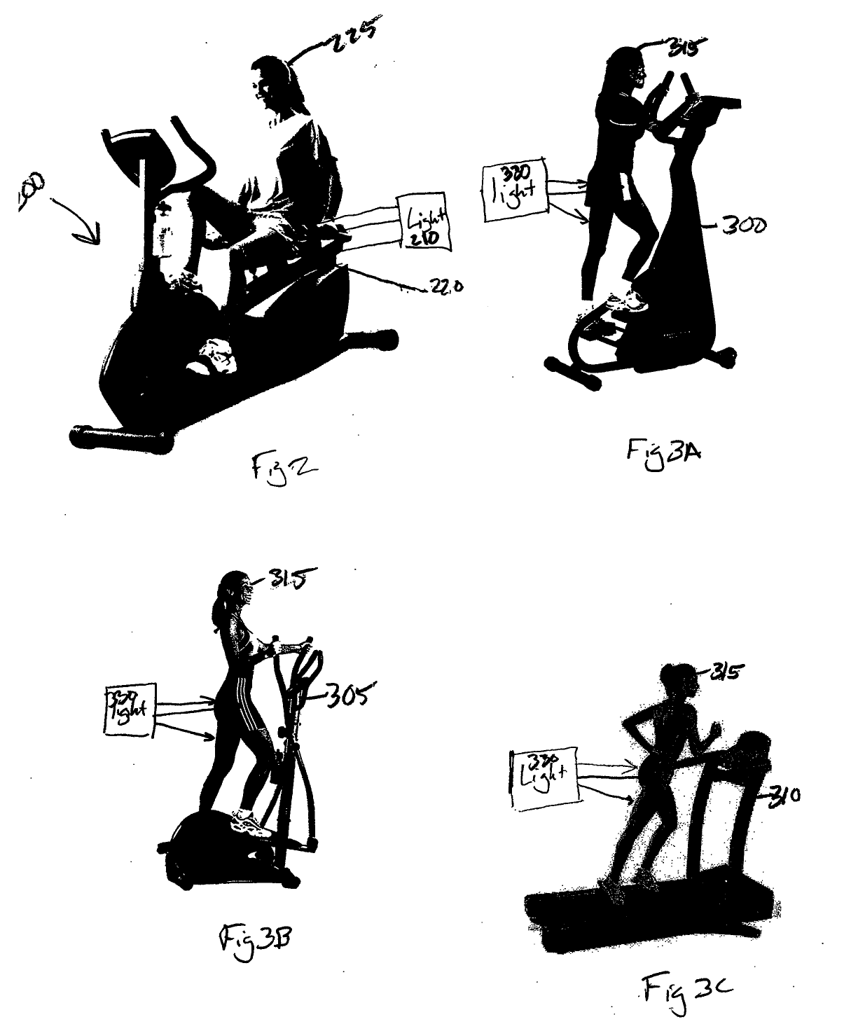

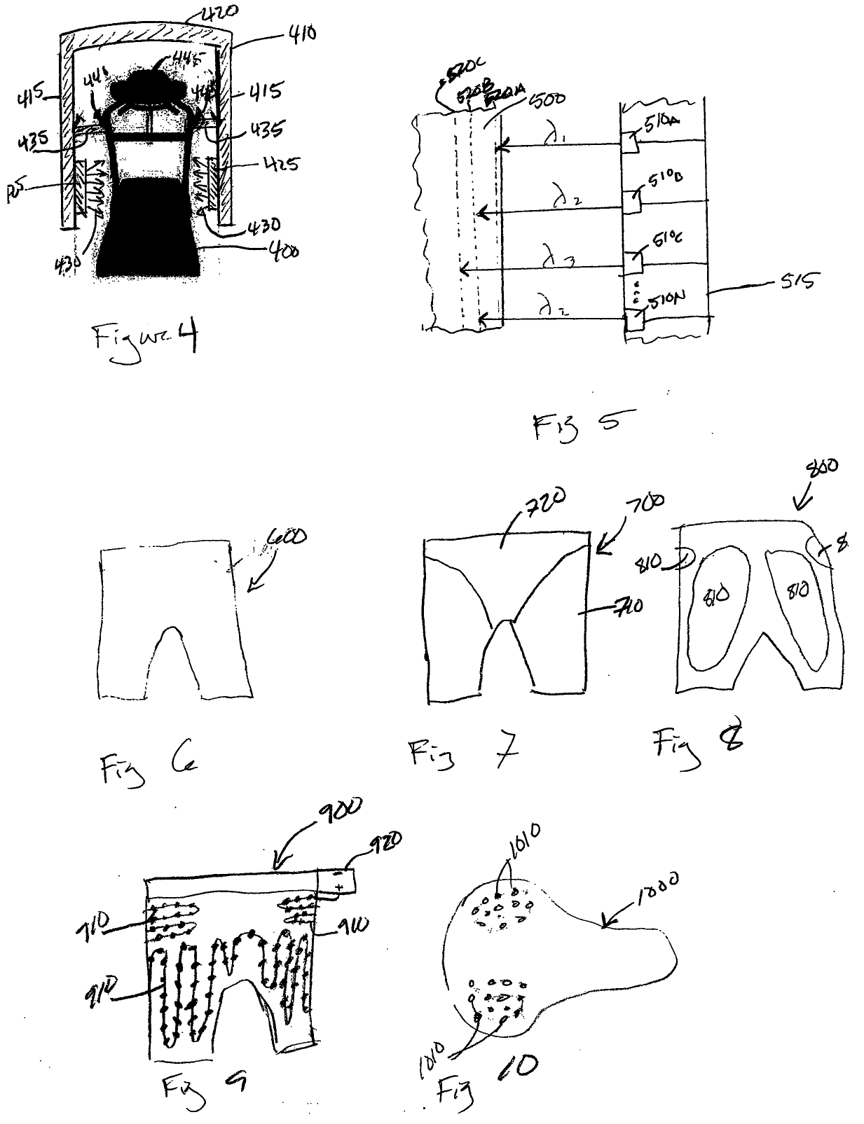

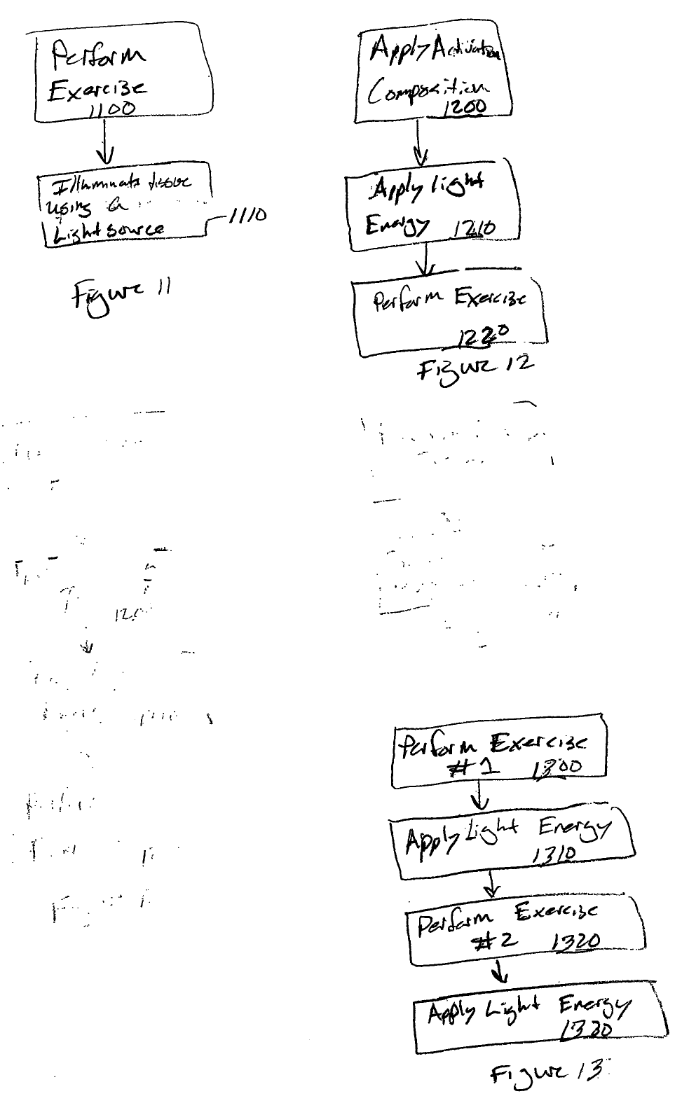

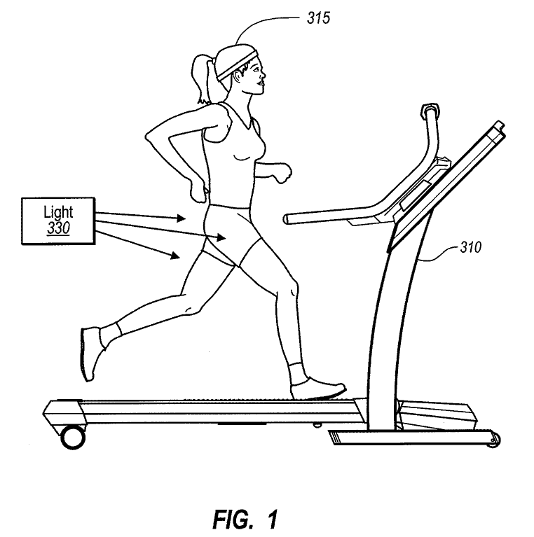

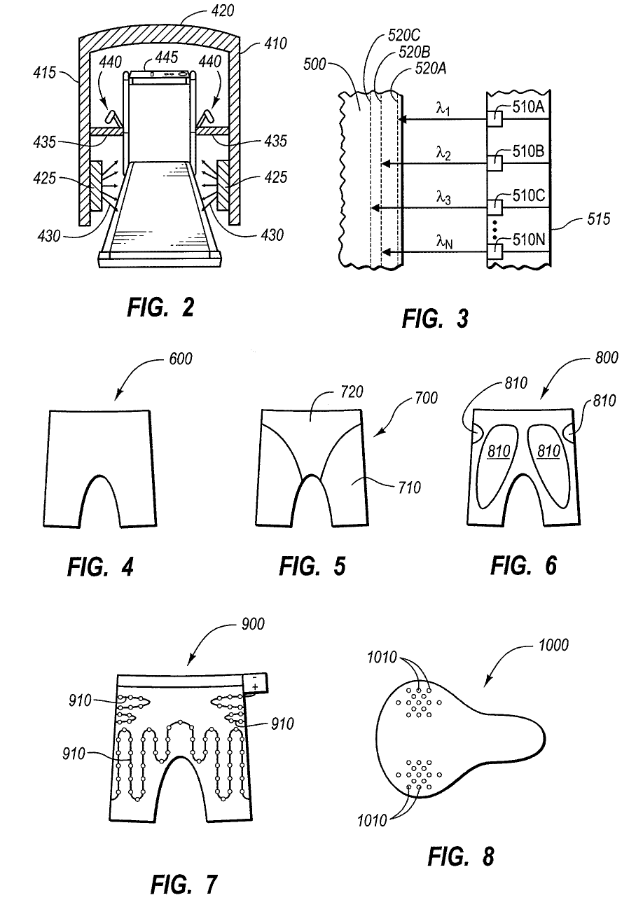

However, not all machines allow the same access for light treatments to the areas in which cellulite is commonly found (i.e., the thighs, buttocks and hips). Referring to FIG. 2, a stationary exercise bicycle is shown. Light 210 for reducing cellulite is applied to the thighs, buttocks, and hips of the person using the exercise bicycle. In this manner, the synergistic effects of both light and exercise can reduce cellulite in these trouble areas. However, as can be appreciated from FIG. 2, the use of a stationary bicycle 200 for application of light to reduce cellulite in the target areas (i.e., the thighs, buttocks, and hips) cannot be easily accessed because of how the person is positioned upon the exercise bicycle. More specifically, the seat 220 of the exercise bicycle and the hunched-over position of the person 225 substantially blocks application of light to much of the rear portion of the person's thighs, buttocks and hips. Thus, the synergistic effects of exercise and light for reduction of cellulite cannot be realized to the same extent as when using other embodiments.

Referring to FIGS. 3A, 3B, and 3C, three examples of exercises are illustrated where the synergistic effects of exercise and light for reduction of cellulite can be more fully realized. FIGS. 3A, 3B, and 3C illustrate examples where light 330 is applied to the thigh, buttocks, and hip regions of a person 315 using a stair stepper 300, an elliptical glider 305, and a treadmill 310 exercise equipment, respectively. As shown, the person 315 is exercising in a more upright position then the person in FIG. 2 and the target areas of the thighs, buttocks and hips are more exposed to the light energy. In many such embodiments as those illustrated in FIGS. 3A, 3B, and 3C, there is no seat, handles, and/or other objects or body parts that obstruct light from focusing on the target areas. Exercises and equipment other than the stair stepping 300, elliptical gliding 305, and treadmill 310 equipment can recognize similar benefits where light is allowed to reach the target areas with less obstruction. Several embodiments also allow the person exercising to exercise in a less hunched over position, as compared to that illustrated in FIG. 2. Thus, the light can be more readily focused on the target areas for treating cellulite, and the person exercising is allowed to be in a free motion and ergonomic position during the exercise. As a result, the synergistic effects of light and exercise can be more effectively realized according to these, and other, embodiments.

B. Separate or Separable Light and Exercise Equipment

Another aspect of several embodiments relates to separate or separable light producing and exercise apparatuses. For example, referring to FIG. 4, a treadmill 400 is shown where the treadmill 400 is releasably attached to a light producing structure 410. The treadmill 400 can be any treadmill type or design. The treadmill 400 can also be a special purpose treadmill that includes mechanical and/or electrical connections for receiving mechanical and/or electrical connections from the light producing structure 410. The light producing structure 410 can include vertical supports 415, and the vertical supports 415 can be coupled together by at least one lateral support 420. The lateral support 420 is not, however, necessary and the vertical supports 415 can be free standing and placed at a location to provide light to the target areas of a person exercising on the treadmill 400.

The vertical supports 415 can include light producing fixtures 425 for producing and directing the light 430 toward a person using the treadmill 400. The light 430 works synergistically with the treadmill 400 to reduce cellulite in the target areas of a person exercising on the treadmill 400. The light producing fixtures 425 can be located and configured to focus light in a particular direction toward the target areas. The light producing structure 410 and/or the treadmill 400 can include connecting portions 435 for mechanically coupling a portion of the treadmill 400 to the light producing structure 410. Connecting portions 435 can connect any portion of the light producing structure 410 to the treadmill 400, and the connecting portions 435 can include releasable engaging mechanisms 440 that can be released to separate the treadmill 400 from the light producing structure 410. The releasable engaging mechanisms 440 can include pins, screws, bolts, friction engagements, or the like, for mechanically engaging a a portion of the connecting portions 435 with the light producing structure 410 and/or the treadmill 400. The releasable engaging mechanisms 440, or other portion of the light producing structure 410 and/or treadmill 400, can also include electronic connections for providing electrical communication of data and/or instructions between electronic components of the light producing structure 410 and the treadmill 400. In this manner the electronic components of the treadmill 400 and the light producing structure 410 can communicate information such as control and operation settings. The treadmill 400 and/or the light producing structure 410 can include displays and/or a user interface to communicate with a person. For example, as shown, the treadmill 400 can have a display 445 to display information for a user.

The releasable engaging mechanism 440 illustrated in FIG. 4 can be separated from the treadmill 400 and releasably attached to a different location on the treadmill 400 and/or to a different type of exercise equipment. For example, the light producing structure 410 can be releasably coupled to one or more locations on an elliptical glider, stair stepper, stationary exercise bicycle, a free weight apparatus, a resistance weight apparatus, or other aerobic or anaerobic exercise apparatus. Thus at least two different types of exercise equipment can be interchangeable and/or separable from the same light producing structure 410, optionally with the ability to attach the light producing structure 410 to multiple locations on the same exercise equipment.

The connecting portions 435 and releasable engaging mechanisms 440 can be omitted. Thus, the light producing structure 410 and the exercise equipment can be entirely separate apparatuses, and the treadmill 400 shown in FIG. 4 can be interchangeable with different types of exercise equipment such as an elliptical glider, a stair stepper, stationary exercise bicycle, a free weight apparatus, a resistance weight apparatus, or other aerobic or anaerobic exercise apparatus by placing the different exercise equipment within the light producing structure 415.

Notwithstanding the foregoing, it should be understood that it is within the scope of the invention to provide exercise equipment in which the light producing structure is permanently attached or affixed to the exercise equipment.

C. Selection of Light Sources

Various light sources are available for generating light, and any light source can be used in any of the embodiments disclosed herein. However, an aspect of several embodiments disclosed herein relates to the use of particular light source(s), or combinations of light sources, that have particular light emitting and operating characteristics which can be particularly advantageous.

Light emitted from different light sources can vary in several characteristics. Examples of characteristics of light which can be varied based on the selection of the light source include intensity (also referred to as brilliance, amplitude, and brightness), frequency (also referred to as wavelength and color), and polarization (or angle of vibration). Light sources can also be selected based on characteristics of transmission, such as control over the direction of transmission and divergence or collimation of the light emission. Light sources can also be selected based on environmental and/or operational characteristics, such as power consumption, light source size, flexibility of placement, consistency of operational characteristics over an extended time period, heat generated by the light source, additional circuitry requirements for operation, and potential failures experienced with the light source. An aspect of several embodiments discussed herein recognizes that selection of a particular light source, or combination of light sources, in combination with exercise can have many advantageous effects.

One type of light source is the light bulb. The term “light bulb” includes incandescent light bulbs, fluorescent lamps, and high-intensity discharge bulbs. The incandescent light bulb uses a glowing wire filament heated to white-hot by electrical resistance, to generate light. The “bulb” or “lamp” is the glass enclosure which keeps the filament in a vacuum, or low-pressure noble gas or a halogen gas in the case of quartz-halogen lamps, in order to prevent oxidation of the filament at high temperatures.

A fluorescent lamp is a type of lamp that uses electricity to excite mercury vapor in argon or neon gas, producing short-wave ultraviolet light. This light then causes a phosphor to fluoresce, producing visible light. Unlike incandescent lamps, fluorescent lamps require additional bulky components and circuitry, such as a ballast, to convert the power supplied to the lamp's fixture into power suitable for the lamp type.

High-intensity discharge (HID) lamps include several types of electrical lights: mercury vapor, metal halide, high-pressure sodium and less common, xenon short-arc lamps. Like fluorescent lamps, HID lamps also require additional bulky circuitry and components, such as a ballast, to start and maintain their operation. Metal halide lamps, in particular, are less efficient than most other lamps but produce an even whiter, more natural light. HID lamps create a relatively large amount of heat, which can be very uncomfortable for a person exercising.

An aspect of several embodiments disclosed herein realizes, however, that in many instances, solid state light sources, such as silicon-based emitters, have better characteristics in combination with exercise for many applications. Two types of silicon based emitters that are particularly advantageous in many of the applications discussed herein are Light Emitting Diodes (LED)s and Laser Diodes (LD)s.

A LED is a semiconductor device that emits incoherent narrow-spectrum light when electrically biased in the forward direction. This effect is a form of electroluminescence. The color of the emitted light depends on the chemical composition of the semiconducting material used, and can be near-ultraviolet, visible or infrared. If the emissive layer material of an LED is an organic compound, it is known as an Organic Light Emitting Diode (OLED).

LEDs can offer several advantages over bulbs when combined with exercise. For example, LEDs are capable of emitting light of an intended color without the use of color filters that other lighting methods often require. Combinations of LEDs can also be produced to create a particular combination of light spectra at selected relative intensities. The shape of the LED package can also allow light to be focused as compared to bulbs and lamps. Incandescent and fluorescent sources often require an external reflector to collect light and direct it in a useable manner. LEDs, on the other hand, emit light in a more defined direction than bulbs and lamps.

LEDs are built inside solid cases that protect them, making them hard to break and extremely durable for better use in combination with robust exercise equipment. LEDs also have an extremely long life span: often twice as long as the best fluorescent bulbs, and twenty times longer than the best incandescent bulbs. LEDs come in extremely small packages allowing for flexibility in placement. Because placement of LEDs is more flexible, more selective placement of LEDs during manufacture of customized apparatus for illuminating a person when exercising is facilitated. In addition, ergonomics in the design can be increased because the person exercising is not limited by the placement constraints of the light source.

LEDs also create much less heat than bulbs, especially when compared to HID lamps. This greatly reduces energy consumption. It also reduces or eliminates the need for bulky cooling fans to maintain patient comfort. LEDs also do not require a ballast like many lamps and bulbs. Because LEDs produce substantially less heat, a person exercising can have a more enjoyable experience exercising without becoming overheated by the light source alone. In addition, because LEDs do not require ballasts, the weight of the light producing structure can be reduced and light source placement options increased. This also eliminates the cost of the ballast(s) and space considerations for accommodating the ballast(s).

Similar to LEDs, lasers offer many of the same, as well as other, advantages over lamps and bulbs. There are many types of lasers which can be used to produce light in the various embodiments illustrated herein. However, a semiconductor laser, or LD, can be particularly advantageous. A LD is a solid-state laser wherein the active medium is a semiconductor p-n junction similar to that found in a light-emitting diode. There are many types of LDs, such as homojunction lasers, double heterostructure lasers, quantum well lasers, separate confinement heterostructure lasers, distributed feedback lasers, VCSELs, and VECSELs. As discussed above, other light sources, such as the incandescent and fluorescent light bulbs, emit photons over a wide spectrum of wavelengths. Most light sources are also incoherent by emitting light in an undefined direction. By contrast, a laser emits light in a narrow, well-defined, polarized, coherent beam of light consisting of a single wavelength. Thus, lasers exhibit many of the benefits of LEDs as compared to lamps and bulbs.

An aspect of several embodiments relates to utilizing LEDs and/or lasers to create light for reducing cellulite. Some of the characteristics of the created light which can be controlled by use of lasers and LEDs include low heat, low power consumption, high light intensity output, control over light wavelength, control over light transmission direction, improved focusing of light on the target areas, extending the life time of the light sources, consistent light output characteristics over time, and the ability to output a single or multiple defined wavelengths of light simultaneously and of controlled intensity.

D. Light Having Multiple Defined Spectra

Multiple spectra of light can be illuminated upon a person exercising to reduce cellulite. Different spectra of light penetrate tissue to different depths. Thus, where different spectra of light are transmitted into skin, the light can reach cellulite at different depths thereby improving circulation and strengthening connective tissues in multiple locations.

LEDs and lasers can be particularly advantageous for such applications because they can be configured to emit a well defined spectrum of light without the need for filters, reflectors, or other passive devices. For example, referring to FIG. 5, tissue 500 is shown as it is illuminated by multiple light sources 510A-N arranged in a vertical array. The multiple light sources 510A-N are supported by a support structure 515 (e.g. part of light producing fixtures illustrated in FIG. 4). The light sources 510A-N illuminate the tissue 500 at different wavelengths, and the light penetrates the tissue to different depths 520A-C depending on the wavelength.

For illustration, referring to FIG. 5, a first light source 510A emits light at a first wavelength, which penetrates the tissue 500 to a first extent 520A. A second light source 510B emits light at a second wavelength, which penetrates the tissue 500 to a second extent 520B. A third light source 510C emits light at a third wavelength, which penetrates the tissue to a greater extent 520C then both the first and second light sources 510A and 510B. A final light source 510N can emit light at the same wavelength as the second light source 510B, which penetrates the tissue 500 to a similar extent as the light emitted from the second light source 510B. Any number and configuration of light sources and wavelengths can be implemented.

Where LEDs or lasers are used to create the light of different spectra, the light can be of any wavelength produced by LEDs or lasers. For example, the light produced will preferably fall within a range of about 300 nanometer (nm) to about 1800 nm, more preferably in a range of about 400 to about 1200 nm, and most preferably in a range of about 600 nm to about 900 nm. The foregoing spectral ranges may be enhanced or broadened using full spectrum light sources in addition to, or instead of, the LEDs or lasers.

Multiple types of light sources can also be used in any of the embodiments. For example, any combination of lamps, bulbs, LEDs, lasers, as well as other light sources can be used. Operational, input, and output characteristics of the different light sources can be combined to obtain the desired results. Thus, any type or combination of light sources can be used herein, but several embodiments recognize that particular light sources, such as LEDs and lasers, can have benefits over lamps and bulbs in many applications. Cycling of the light outputs can also be conducted.

E. Clothing and Equipment Designs

Clothing designs can improve the application of light to tissue to reduce cellulite. One aspect that several embodiments recognize is that the target areas are located in regions of the bodies that may be particularly embarrassing for people to expose. Thus, several embodiments relate to clothing for exposing the tissue to illuminated light.

Several embodiments include clothing that is at least partially transmissive to light. For example, referring to FIG. 6, a piece of clothing, in this example a pair of shorts 600, is illustrated. The fabric from which at least a portion of the pair of shorts 600 is made is selected for its light transmissive properties. The fabric of the shorts 600 can be made from a particular material selected to transmit light. The fabric of the shorts 600 can also be made by a particular process, such as a particular weave or knit fabrication process that creates small voids or pores in the fabric to allow at least a portion of the light to be transmitted through the material of the shorts 600. Other methods for increasing the amount of light that is transmitted through the shorts 600 can also be implemented.

Clothing can also include different portions of the clothing having different relative transmissive properties. For example, the clothing can have portions of relatively high transmissive properties in the regions of the thighs, buttocks, and hips areas, and other portions with material that is relatively less transmissive to light in other areas of the clothing. For example, referring to FIG. 7, a pair of shorts 700 is illustrated with two materials having different light transmissive properties. A first portion 710 of the shorts 700 can be made from a first material having properties that are more transmissive to light than a second material. The first material can be located on the shorts 700 in areas where cellulite is more likely to be found, such as the thighs, buttocks, and hips. A second portion 720 of the shorts 700 can be made of a second material that is less transmissive to light than the first material.

More than two different materials with different light transmission properties can be used and different lines of clothing can be produced to target different portions of the body. For example, a pair of shorts (or, e.g., pants) can be produced where only the material over the thighs is made of a more transmissive material so as to target only that portion of the body.

Clothing can also have portions of the fabric removed to expose particular portions of the body. For example, referring to FIG. 8, a pair of shorts 800 is illustrated where portions 810 of the shorts over the back of the thigh and buttocks can be removed to expose that portion of the body. In this manner, people can wear the clothes in gyms, spas, at home, and at other locations without overtly showing skin, thus reducing the concern that they may be seen without clothing because they need to expose their skin to the light for reduction of cellulite.

Clothing can also incorporate light sources. For example, clothing can have light sources embedded in the clothing or attached to the clothing. Light sources that are small and that operate at relatively cool temperatures (such as LEDs and LDs) may be particularly advantageous for this application. Clothing can incorporate arrays of lights for illuminating the skin located beneath the clothes. The clothes can be worn during exercise to achieve the effects of reducing cellulite discussed above. Thus, the clothing can be made from a material that is comfortable during exercise but still provides the light illuminating benefits to the skin.

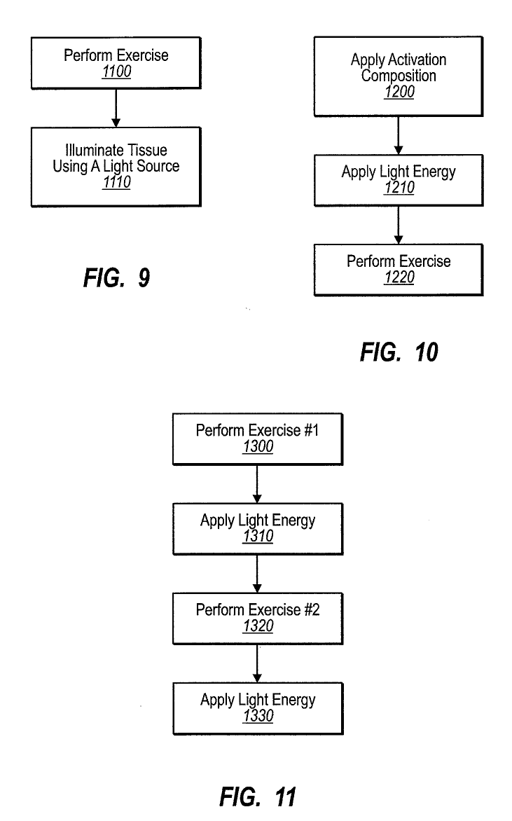

The clothing can be designed to at least partially hide the light sources from view by other persons such that the clothing appears substantially similar to other clothing without the incorporated light sources. The light sources can be incorporated in the clothing to target particular areas, such as the thighs, buttocks, and hips. For example, referring to FIG. 9, an illustration of the back side of a pair of shorts 900 is shown. The shorts 900 incorporate arrays of light sources 910 (e.g. lasers and/or LEDs) for illuminating the tissue under the shorts 900. The location of the arrays of light sources 910 can vary depending on the desired target locations for illumination by light.

Current for driving the light sources can be supplied by any means. For example, a battery 920 can be carried by the person wearing the shorts 900. In this embodiment, low power consumption light sources are particularly advantageous. The shorts 900 can also receive power from an external power source, such as a wall socket or separate battery, which can be connected to the light sources by a cord or other electrical connection. The battery 920 can be rechargeable or replaceable. Clothing incorporating light sources can also include control circuitry for controlling output characteristics of the various light sources, such as intensity, relative intensity between arrays of light sources, active spectra, etc. Thus, the clothing can also include a user interface and/or display for allowing a user to input control parameters for any of the light sources.

In the instance that the type of light illuminating the areas of the person performing exercise is subject to a safety standard (such as an eye safety standard for lasers), the material of the clothing can be selected for its non-transmissive properties to the light produced by the light source(s). For example, where a pair of shorts (such as the shorts shown in FIG. 9) includes lasers, the material of the shorts can be selected to prevent transmission of the light to the outside of the shorts. In this manner, any potentially harmful transmission from the light source (e.g., to an eye) can be prevented.

Exercise equipment and sporting goods can also incorporate light sources or have light sources attached to the equipment. For example, referring to FIG. 10, a bicycle seat 1000 incorporating light sources 1010 is shown according to an exemplary embodiment. The light sources 1010 of the seat can illuminate a buttocks portion of a person exercising using the bicycle seat 1000. The equipment incorporating light source(s) can be any equipment. Equipment incorporating light source(s) where the equipment is located particularly close to, or in contact with the target areas (such as the bicycle seat of FIG. 10), are particularly advantageous because the light produced by the light source(s) can be more easily focused on the target areas where cellulite is found. The light sources illustrate in FIG. 10 can be incorporated into, or attached to, the seat. A power source for driving the light sources can be provided by a batteries or an external source, such as a wall socket.

Light sources can be incorporated into other sporting goods that are intended to aid or facilitate exercise (e.g., aerobic exercise). Examples include, but are not limited to, bicycles, snow skiing clothing, rowing seats, jogging shorts, hiking clothing, athletic team uniforms, and the like.

F. Hydration

Several embodiments relate to combining hydration techniques along with light treatments and/or exercise to reduce cellulite. Proper hydration can help facilitate the reduction of cellulite. Hydration can also improve blood circulation and reduction of fat from the connective tissue. The fat removed from the connective tissue by the blood circulation can be received by the lymph nodes and naturally processed by the body. Thus, proper hydration can have a synergistic effect when implemented along with light treatment and/or exercise to reduce cellulite.

It is within the scope of the invention to recommend that a patient use oxygenated water, which contains a higher content in parts per million of oxygen than ordinary water. Oxygenated water is believed by some to increase the blood oxygen content for increased fat metabolism. One example of oxygenated water is manufactured by increasing oxygen stability in water through a process that attaches oxygen to sodium chloride molecules.

G. Handheld Light Sources

Light sources for reducing cellulite can be incorporated into handheld devices. These handheld devices can be used during exercise to apply light to target areas, such as the thighs, buttocks, and hips. The handheld light sources can be battery operated, in which case low power consumption light sources that operate at low temperatures are advantageous. Handheld light sources can also receive power from external sources, such as a wall socket. Any of the embodiments described herein can operate at any voltage and current, such as standard 110 volts or 220 volts wall sockets or other standard voltages and currents received from batteries.

H. Other Options for Cellulite Reduction

It is within the scope of the invention to combine light treatment with electro-muscle stimulation (e.g., devices used to stimulate the abdominal muscles or other devices that can cause muscles to contract using electrical stimulation instead of pure physical exertion). Light treatment may also be combined with heat producing stimulation, such as a sauna belt.

### II. Activation Compositions

It is within the scope of the invention to utilize light activation compositions for increasing the benefits of the light radiation for reducing cellulite. The compositions can be creams, ointments, lotions, treatments, or other compositions that can be applied to a person's skin during illumination by a light source to reduce cellulite. For example, the activation composition can include photoreceptive properties, such as including colorants, pigments, chemical receptors (such as ethylenediaminetetraacetic acid (EDTA)), or a composition having a selected pH to increase receptiveness of the person illuminated by the light. The activating compositions can increase the effectiveness of the absorption of light by the target areas. For example, the compositions can increase the amount of light that is absorbed by the skin. The compositions can also include compounds for increasing the effectiveness of the light on the skin and tissue by increasing the strengthening the connective tissues or breaking down the fat. The compositions can also include compounds for tightening skin or otherwise reducing the appearance of cellulite. The compositions can also include natural ingredients that help eliminate or reduce toxins trapped beneath the skin that can combine with trapped fat to create cellulite. The person exercising can selectively apply the compositions to the target areas to more closely target the effects of the light illuminated thereon. The pH of the composition can be selected to reduce pigment (e.g., a whitening composition).

The activation compositions for increasing the effectiveness of the light treatments can include other compositions for firming, debrading, desensitizing, sensitizing, exfoliating, and stimulating skin to further reduce cellulite.

### III. Combinations

The various teachings set forth herein can be combined in any manner. For example, a person can apply the activation composition for light treatment during exercise to reduce cellulite. A person can also exercise while wearing the clothing discussed above over their skin where activation compositions have been applied to the skin (or selected areas of the skin). The teachings of proper hydration discussed above can also be combined with any of the other teachings herein. The clothing can also include, or be treated with, compositions for enhancing the effects of the light illuminated. For example, the clothing can include, or be treated with, compositions to intensify the light illuminate on certain areas (such as the thighs, buttocks, and hips) of a person wearing the clothing.

For example, referring to FIG. 11, a method for reducing cellulite is illustrated. The method can include performing an exercise (1100) and illuminating tissue using light produced by a light source (1110). The light source can include a semiconductor material, such as silicon. The exercise can be conducted on a treadmill, a stationary bicycle, a stair stepping device, an elliptical glider, a free weight exercise device, and/or a resistance weight exercise device, or any other exercise device. The light source can include a LD or a LED. The light can include multiple spectra. A person can perform the exercise in a substantially upright position to expose the person's thighs, buttocks, and hips without obstruction, the exercise can be performed while the person is wearing clothing, where the clothing includes multiple portions of different relative transmissive characteristics to light, or to light of a particular spectrum. The clothing can also have portions of the clothing over the target areas removed to reveal the tissue for illumination by the light in these areas. A composition, as described above, can be applied to the person performing the exercise prior to the person performing the exercise, and the composition can be applied only in the target areas. The light source can be permanently or releasably attached to the exercise equipment, or the light source may not be attached to the exercise equipment at all. The light source can be incorporated within, or attached to, clothing, such as shorts, or a piece of the exercise equipment, such as a seat. Methods of hydration can also be incorporated.

Referring to FIG. 12, a block diagram illustrating a method for reducing cellulite is shown. An activation composition can be applied to a person (1200) and the person can be illuminated by light energy (1210). The activation composition can be any activation composition discussed above. The activation composition may selectively applied to targeted areas of the person. The person can also perform an exercise (1220). The illumination and exercise can be performed simultaneously. The exercise can be performed using a treadmill, a stationary exercise bicycle, a stair stepper, an elliptical glider, a free weight exercise device, a resistance exercise device, or any other exercise device. The devices can be interchangeable and the person can perform multiple different exercises while being illuminated.

Referring to FIG. 13, a block diagram illustrating a method for reducing cellulite is shown. A person can perform a first exercise (1300). The person can be illuminated during the first exercise (1310). The person can perform a second type of exercise (1320). The second type of exercise can be performed using a different piece of exercise equipment. Light energy can be applied to the person during the second exercise (1330). For example, the two exercises can be performed using different exercise equipment that are interchangeable with a single device for illuminating the person performing the exercises.

Any of the steps and/or acts discussed above (e.g., in reference to FIGS. 11, 12, and/or 13) can be practiced in any combination or sequence. Steps and/or acts can also be eliminated (e.g., from FIGS. 11, 12, and/or 13). The embodiments discussed herein can be combined with various other physical therapy and muscular ailment treatments. In many instances, the combination of the teachings herein with other treatments and remedies can have similar synergistic effects.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for reducing cellulite comprising:
performing an exercise; and
illuminating cellulite-containing tissue using light produced by a light source comprising a semiconductor material.

2. A method according to claim 1, wherein the exercise is performed using exercise equipment, the exercise equipment comprising at least one of:
a treadmill;
a stationary bicycle;
a stair stepping device;
an elliptical glider;
a free weight exercise device; or
a resistance weight exercise device.

3. A method according to claim 1, wherein the light source comprises at least one of:
a light source comprising silicon;
a laser; or
a laser diode.

4. A method according to claim 1, wherein the light includes light of multiple substantially defined spectra.

5. A method according to claim 1, wherein a person exercising is exercising in a substantially upright position such that the person's thighs, buttocks, and hips are substantially exposed to the light without substantial obstruction.

6. A method according to claim 1, wherein a person performing the exercise is wearing clothing, the clothing comprising:
a first portion including a first material; and
a second portion adjacent to the cellulite-containing tissue and including a second material, wherein the second material is more transmissive to the light than the first material.

7. A method according to claim 1, wherein a person performing the exercise is wearing clothing, the clothing comprising:
a removed portion adjacent to the cellulite-containing tissue.

8. A method according to claim 1, further comprising:
applying to skin of the cellulite-containing tissue a composition that increases reception of the light.

9. A method according to claim 8, wherein the composition comprises at least one of:
a colorant;
a pigment;
a chemical receptor;
ethylenediaminetetraacetic acid (EDTA); or
a selected pH for enhancing reception of the light.

10. (canceled)

11. (canceled)

12. (canceled)

13. (canceled)

14. A system for reducing cellulite comprising means for performing the method of claim 1.

15. A system according to claim 14, wherein the system comprises exercise equipment which comprises at least one of:
a treadmill;
a stationary bicycle;
a stair stepping device;
an elliptical glider;
a free weight exercise device; or
a resistance weight exercise device.

16. (canceled)

17. (canceled)

18. (canceled)

19. A system according to claim 14, wherein the light source forms part of a piece of clothing.

20. (canceled)

21. Clothing for use in reducing cellulite, comprising:
a fabric;
a light source comprising a semiconductor material, attached to the fabric, and configured to illuminate cellulite-containing tissue of a person wearing the clothing.

22. Clothing according to claim 21, wherein the light source comprises at least one of silicon, a light emitting diode, or a laser.

23. Clothing according to claim 21, wherein the light source comprises an array of light sources incorporated with, or attached to, the clothing.

24. Clothing according to claim 21, wherein the array of light sources are located within the clothing to illuminate the thigh, buttocks, or hips areas of a person wearing the clothing.

25. Clothing according to claim 21, further comprising an activation composition incorporated into the fabric.

26. (canceled)

27. (canceled)

28. (canceled)

29. (canceled)

30. (canceled)

31. Equipment for reducing cellulite, comprising:
exercise equipment, comprising:
a treadmill;
a stationary bicycle;
a stair stepping device;
an elliptical glider;
a free weight exercise device; or
a resistance weight exercise device; and
an illumination source, the illumination source being disposed so as to illuminate the buttocks and/or thigh of a person using the exercise equipment.

32. Equipment according to claim 31, further comprising structure supporting the illumination support, wherein the structure is structurally connected to the exercise equipment.

33. Equipment according to claim 31, further comprising clothing supporting the illumination source, wherein the clothing supports the illumination source at a location adjacent to the buttocks and/or thigh of the person using the exercise equipment.
